(19) United States
(12) Patent Application Publication
(10) Pub. No.: US 2018/0326124 A1
(43) Pub. Date: Nov. 15, 2018

(54) Bio-Material Composition and Methods of Use

(71) Applicant: Bone Solutions, Inc., Colleyville, TX (US)

(72) Inventors: Drew Diaz, Colleyville, TX (US); Frankie L. Morris, Colleyville, TX (US); Brandon Roller, Colleyville, TX (US)

(21) Appl. No.: 15/978,559

(22) Filed: May 14, 2018

Related U.S. Application Data
(60) Provisional application No. 62/505,220, filed on May 12, 2017.

(51) Int. Cl. A61L 27/58; A61L 27/04; A61L 27/12; A61L 27/54; A61L 27/20; A61L 27/18

(52) U.S. Cl. CPC A61L 27/58; A61L 27/047; A61L 27/12; A61L 2300/406; A61L 27/20; A61L 27/18; A61L 2430/02; A61L 27/54

(57) ABSTRACT

The present invention relates to a bio-material composition comprising: a dry potassium phosphate based mixture comprising (a) MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1; and (b) an aqueous solution, wherein the dry potassium phosphate based mixture is mixed with the aqueous solution forming a reabsorbable bio-material slurry, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof.

### RELATED APPLICATIONS

This application claims priority to U.S. Provisional Application No. 62/505,220 entitled “Bio-Material Composition and Methods of Use,” filed on May 12, 2017, the contents of which are hereby incorporated by reference in its entirety.

### FIELD OF THE INVENTION

The present invention relates to a bio-material composition and methods of use. Embodiments of the formed bio-material have significantly increased porosity, increased reabsorbability, and/or increased cohesion compared to existing formulations.

### BACKGROUND

Unless otherwise indicated herein, the materials described in this section are not admitted to be prior art to the claims in this application.

Increasing numbers of sports, age, and trauma related injuries like broken bones, worn out joints, and torn ligaments have heightened the demand for bio-materials capable of treating orthopedic injuries. In response, companies have developed bone cements to attach various objects to bone, and bone fillers capable of treating bone fractures and other bone defects. There is also a need for a bio-material capable of stimulating bone formation and growth. Most existing bio-materials are made of calcium phosphates that promote significant new bone formation or relatively inert hardening polymers like polymethylmethcrylate (“PMMA”) that are poorly biocompatible.

U.S. Pat. No. 5,968,999 issued to Ramp et al, describes a PMMA based bone cement composition useful for orthopedic procedures. Unfortunately, PMMA-based bio-materials release considerable amounts of heat to the surrounding bone during the curing process causing cell death. The resulting materials shrink during setting and have poor resistance to fracture. PMMA biomaterials also possess slow rates of bio-absorption and poor bio-compatibility due to the release of a toxic monomer into the blood stream. There is little evidence that PMMA based materials promote any significant new bone formation.

A number of calcium phosphate based compositions have been developed as biomaterials in recent years. For example U.S. Pat. No. 6,331,312 issued to Lee et al., discloses an injectable calcium phosphate based composite useful as a bone filler and cement. The disclosed material is bio-resorbable and is designed for use in the repair and growth promotion of bone tissue as well as the attachment of screws, plates and other fixation devices. Lee's composition does not expand while setting and does not promote significant new bone formation. Many existing calcium phosphate based fillers and cements have high molar ratios of Ca to P making them poorly reabsorbable. Furthermore, a recent FDA release warns of serious complications from the use of existing calcium phosphate based bone fillers in treating fractures of the spine (FDA Public Health Web Notification, “Complications Related to the Use of Cement and Bone Void Fillers in Treating Compression Fractures of the Spine,” originally published Oct. 31, 2002, updated, May 27, 2004.)

U.S. Pat. No. 9,078,884 discloses a bio-material composition and method for spinal fusion, the contents of which are incorporated by reference herein in their entirety. U.S. non-provisional patent application Ser. No. 14/621,920 discloses a multi-purpose bio-material composition, the contents of which are incorporated by reference herein in their entirety. U.S. non-provisional patent application Ser. No. 14/797,183 discloses a bio-material composition and method of use, the contents of which are incorporated by reference herein in their entirety.

### SUMMARY

The present invention comprises a bio-material composition and method of producing such, wherein one or more of the embodiments formed has significantly increased porosity, increased reabsorbability, and increased cohesion compared to existing formulations.

The present invention comprises a bio-material composition comprising (a) a dry potassium phosphate based mixture comprising MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1; and (b) an aqueous solution, wherein the dry potassium phosphate based mixture is mixed with the aqueous solution forming a reabsorbable bio-material slurry, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof, wherein the reabsorbing agent provides unexpectedly improved reabsorption, porosity, and cohesion of the bio-material composition.

In one particular example, the bio-material composition comprises mono potassium phosphate between about 44-61% of the total composition. The mono potassium phosphate provides faster absorption of the composition. The bio-material composition also comprises tricalcium phosphate between about 4-9% of the total composition. The tricalcium phosphate provides additional strength and platform for bone growth. The bio-material composition also comprises mono sodium phosphate between about 4-9% of the total composition, which helps control ion release. The bio-material composition also comprises magnesium oxide between about 30-45% of the total composition, which may be hard burned to provide controlled reactivity. The MgO enables the composition to be reabsorbed faster compared to existing calcium bone void fillers. The MgO stimulates osteoblast activity as osteoblasts use magnesium as fuel in the bone formation process. The bio-material composition also comprises sugar between about 0-5% of the total composition, which increases porosity and further helps control the reaction. The bio-material composition also comprises phosphoric acid between about 0-5% of the total composition, which helps break down MgO to generate more phosphate. The bio-material composition also comprises Hydroxypropyl methyl cellulose (HPMC) between about 0-10% of the total composition, which helps provide improved handling characteristics. The bio-material composition also comprises Carboxymethylcellulose (CMC) between about 0-10% of the total composition, which helps provide improved handling characteristics. The bio-material composition also comprises a poloxamer between about 0-10% of the total composition, which helps provide improved handling characteristics. Various combinations of the above components and percentages are possible as well.

The bio-material composition may be applied to various sites including but not limited to sites on or adjacent to bone; sites on, in, or adjacent to a cartilage; sites in, on, or proximate to bone or cartilage, and bone or cartilage contacting surfaces of implant devices. The bio-material composition may be applied directly to bone defects acting as a bone filler, bone cement, delivery device, bone graft and/or general binder matrix. Alternatively, the bio-material composition may be used in conjunction with various fixation devices such as screws and plates.

In another example, the present invention comprises a method for producing a bio-material with increased porosity and reabsorption characteristics, the method comprising: (a) supplying a dry potassium phosphate based mixture comprising: MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1, and (b) mixing the dry potassium phosphate based mixture with an aqueous solution forming an reabsorbable bio-material slurry.

In yet another example, the present invention comprises a method for back-filling a bone defect void using a bio-material with increased porosity and reabsorption characteristics, the method comprising: (a) removing a bone defect from a bone to create a void, (b) mixing a dry potassium phosphate based mixture with an aqueous solution to form a reabsorbable bio-material slurry, wherein the dry potassium phosphate based mixture comprises MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof, and (c) back-filling the void with the reabsorbable bio-material slurry, wherein the reabsorbable bio-material slurry increases osteoblast activity in the bone to help maintain the structure of the bone.

These as well as other aspects, advantages, and alternatives, will become apparent to those of ordinary skill in the art by reading the following detailed description, with reference where appropriate to the accompanying drawings.

### DETAILED DESCRIPTION

Exemplary devices and systems are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting.

As used herein, with respect to measurements, “about” means+/−5%.

As used herein, “Osteoconductive” is the ability of material to serves as a scaffold for viable bone growth and healing.

As used herein, “Osteoinductive” refers to the capacity to stimulate or induce bone growth.

As used herein, “Biocompatible” refers to a material that does not elicit a significant undesirable response in the recipient.

As used herein, “Bioresorbable” is defined as a material's ability to be resorbed in-vivo through bodily processes. The resorbed material may be used the recipients body or may be excreted.

### I. PREPARING/SUPPLYING THE DRY MIXTURE

The dry mixture of the invention generally comprises: magnesia, potassium biphosphate, and a calcium tricalcium phosphate, wherein the weight percent ratio of potassium biphosphate to magnesia is between about 3:1 and 1:1. In one or more preferred embodiments the dry mixture also comprises a sugar and/or a mono-sodium phosphate. It may be preferable to produce the dry mixture in advance. After it is prepared it should be stored in a sterile environment and more preferably a sterile and sealed container or packaging.

The dry components of the mixture can be mixed using a variety of methods including hand mixing or machine mixing. One method for mixing, sizing, and homogenizing the various powders is via vibratory milling. Another homogenization method utilizes a ribbon mixer wherein the particles are ground to a fine size. It may be preferable to mix the dry components again on-site before the addition of the activating aqueous solution.

The magnesia of the composition is optionally subjected to a calcination and thermal decomposition process. Calcination of the MgO is a treatment process in the absence or limited supply of air or oxygen applied to ores and other solid materials to bring about a thermal decomposition. Thermal decomposition, or thermolysis, is a chemical decomposition caused by heat. The decomposition temperature of a substance is the temperature at which the substance chemically decomposes. The reaction is usually endothermic as heat is required to break chemical bonds in the compound undergoing decomposition. In other words, this process allows the MgO to break down and turn into a hydrate so it will be reabsorbed by the body.

Calcination durations and temperatures are determined empirically, depending on the final characteristics and setting times desired. In some embodiments calcination temperatures of up to about 1300° C. for up to several hours are used, although calcination can be varied. Those of ordinary skill in the art of preparation of similar bone compositions could routinely determine the appropriate calcination conditions to achieve the desired properties.

In addition to the aqueous forms, the composition of the present invention can be a gel comprising the dry mixture.

Generally, pharmaceutical grade compounds are utilized when available. Sterilization of the components, utensils, solutions, etc., used to make and apply the slurry may be required using suitable sterilization techniques known in the art including but not limited to chemical sterilization techniques, such as gassing with ethylene oxide, and sterilization by means of high-energy radiation, usually γ radiation or β radiation.

While the formulations described in Section IV below and weight percentages are the preferred proportions, a range of dry constituents can also be used. For example, a suitable range for the potassium biophosphate (i.e., MKP) is generally between about 20-70 weight percent, preferably between about 40-65 weight percent. In some situations and/or embodiments it is preferable to use the potassium phosphate at a range between about 40-50 weight.

A suitable range for the magnesia (i.e., MgO) is generally between about 10-60, preferably between 10-50, and even more preferably between 30-50 weight percent. In some situations and/or embodiments between about 35 and 50 weight percent can be used.

Tricalcium phosphate (preferably a tricalcium apatite) and other calcium phosphates can be added in various weight percentages. The calcium containing compound(s) is/are preferably added at about 1-15 weight percent, more preferably between about 1-10 weight percent. Higher percentages can be employed in certain situations.

Sugars (and/or other carbohydrate containing substances) are generally present at weight percent between 0.5 and 20, preferably about 0.5-10 weight percent of the dry composition. Suitable sugars include sugar derivatives (i.e., sugar alcohols, natural and artificial sweeteners (i.e., acesulfame-k, alitame, aspartame, cyclamate, neohesperidine, saccharin, sucralose and thaumatin), sugar acids, amino sugars, sugar polymers glycosaminoglycans, glycolipds, sugar polymers, sugar substitutes including sugar substitutes like sucralose (i.e., Splenda®, McNeil Nutritionals LLC, Ft. Washington, Pa.), corn syrup, honey, starches, and various carbohydrate containing substances.

Typically an antibiotic, antibacterial or antiviral agent is added at a weight percent of less than about 20 weight percent of the dry composition, preferably between about 0.5 and 10 weight percent, more preferably between about 1 and 5 weight percent. Any antibiotics typically used in joint replacement and repair surgeries can be used.

Water (or another aqueous solution) can be added in a large range of weight percents generally ranging from about 15-40 weight percent, preferably between about 20-35 weight percent and even more preferably between about 28-32 weight percent. It was found that a saline solution may be used. An exemplary saline solution is a 0.9% saline solution.

### II. FORMING A REABSORBABLE BIO-MATERIAL SLURRY

The dry mixture is preferably activated on-site. Activation comprises mixing the dry composition with an aqueous solution (such as in a sterile mixing vessel to a form a reabsorbable bio-material slurry. Water (e.g., sterile water (or other sterile aqueous solution, e.g., i.e., slight saline solution) is generally added up to about 40% of the dry weight, although the amount of water can be adjusted to form a bio-material of varying viscosity. In one embodiment, the mixing vessel and any utensils are sterilized prior to use. Various mixing vessels can be used including but not limited to a sterile medicine cup, bowl, dish, basin or other sterile container.

Mixing can be achieved by a variety of techniques used in the art including hand and electric/automated mixing. One preferred method is to hand mix with a sterile spatula or other mixture utensil. The reabsorbable bio-material slurry is typically hand mixed for between about 1-10 minutes, although mixing times can be adjusted depending upon conditions and mixing means.

It is possible to mix the slurry using manual hand mixers like the Mixevac III from Stryker (Kalamzoo, Mich.) or an electric bone mixer like the Cemex Automatic Mixer from Exactech (Gainesville, Fla.).

The reabsorbable bio-material slurry can be created in injectable, paste, puddy and other forms. Because the slurry is produced at the user site, the consistency of the material can be manipulated by varying the amount of water added to the dry mixture. Increasing the water content generally increases the flowability while decreasing the water content tends to thicken the slurry.

Working times can be increased or decreased by varying the temperatures of bio-material components. Higher temperature components tend to react and set quicker than cooler components. Thus regulating the temperature of the water (or other reactants) can be an effective way to regulate working time.

The inventor has found that the use of a phosphoric acid solution instead of water increases the bonding strength of the material. The molarity of the phosphoric acid can vary, as long as the eventual pH of the slurry is not hazardous to the patient, or contraindicative to healing.

### III. APPLYING THE REABSORBABLE BIO-MATERIAL SLURRY TO THE SITE

Once the reabsorbable bio-material slurry has been formed it is applied to (and optionally also around) the site of desired cartilage growth. The slurry can be applied to the site in a number of ways including but not limited to spreading an amount of the material to the site using a sterile spatula, tongue blade, knife or other sterile implement useful for spreading a paste or puddy-like material. In some situations it may be preferable to use a relatively thick consistency like a paste or puddy when applying the activated slurry, since such consistencies tend to stick to bone and other surface more easily than thinner ones. If an injectable formation is desired, it can be applied using a syringe or other similar device.

### IV. EXEMPLARY FORMULATIONS OF THE DRY MIXTURE

Exemplary formulations of the dry mixture include the following:

Formulation I *

| | |
|---|---|
| Mono-potassium phosphate (i.e., KH₂PO₄) | 61% |
| Magnesia (calcined) | 31% |
| Ca₁₀(PO₄)₆(OH)₂ | 4% |
| Sucrose C₁₂H₂₂O₁₁ (powder) | 4% |

* All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between about 20-35 weight percent.

Formulation II*

| | |
|---|---|
| KH₂PO₄ | 54% |
| MgO (calcined) | 33% |
| Calcium-containing compound | 9% (whereby the compound is |
| | Ca₁₀(PO₄)₆(OH)₂) |
| Sucrose C₁₂H₂₂O₁₁ (powder) | 4% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between about 20-35 weight percent.

Formulation III*

| | |
|---|---|
| KH₂PO₄ | 44% |
| MgO (calcined) | 44% |
| Calcium-containing compound | 8% (whereby the compound is |
| | Ca₁₀(PO₄)₆(OH)₂ or CaSiO₃, |
| Sucrose C₁₂H₂₂O₁₁ (powder) | 4% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between about 20-35 weight percent.

Formulation IV*

| | |
|---|---|
| Potassium phosphate (i.e., KH₂PO₄) | 44% |
| MgO (calcined) | 41% |
| Ca₁₀(PO₄)₆(OH)₂ | 8% |
| Sucrose C₁₂H₂₂O₁₁ (powder) | 4% |
| Mono-sodium phosphate (MSP) | 3% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between about 20-35 weight percent, more preferably between about 28-32 weight percent.

Formulation V*

| | |
|---|---|
| KH₂PO₄ | 45% |
| MgO (calcined) | 45% |
| Calcium-containing compound | 9% |
| Sucrose C₁₂H₂₂O₁₁ (powder) | 1% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between about 20-35 weight percent.

Formulation VI*

| | |
|---|---|
| KH₂PO₄ | 45% |
| MgO (calcined) | 45% |
| Ca₁₀(PO₄)₆(OH)₂ | 8% |
| Sucralose | 2% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between 20-35 weight percent.

Formulation VII*

| | |
|---|---|
| KH₂PO₄ | 61% |
| MgO (calcined) | 32% |
| Ca₁₀(PO₄)₆(OH)₂ | 4% |
| Collagen | 1.5% |
| α-Ca₃(PO₄)₂ | 1.5% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between 20-35 weight percent.

Formulation VIII*

| | |
|---|---|
| KH₂PO₄ | 50% |
| MgO (calcined) | 35% |
| Ca₁₀(PO₄)₆(OH)₂ | 7% |
| β-Ca₃(PO₄)₂ | 3% |
| Dextrose | 5 |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between 20-35 weight percent.

Formulation IX*

| | |
|---|---|
| KH₂PO₄ | 54% |
| Phosphoric Acid | 4% |
| Metal oxide | 32% (wherein the metal oxide is MgO, |
| | ZrO, FeO or combination thereof), |
| Ca₁₀(PO₄)₈(OH)₂ | 7% |
| Thrombin | 3% |

*All values are weight percentages

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between 20-35 weight percent.

Formulation X*

| | |
|---|---|
| KH₂PO₄ | 45% |
| MgO (calcined) | 45% |
| Ca₁₀(PO₄)₆(OH)₂ | 10% |

Blood or bone marrow derived product (including but not limited to whole blood, (PRP) platelet rich plasma, (BMA) bone marrow aspirate, (BMC) bone marrow concentrate), or a modified solution (including but not limited to mixture of sterile water and Sodium Chloride or sterile water and Sodium Chloride/Sodium Phosphate) is added up to about 40 weight percent of the dry formulation, preferably between 20-35 weight percent.

For some embodiments (i.e., formula III) it has been found that adding water at a weight percent of about 37 weight percent produces a creamy textured material that is extremely easy to work with has excellent adhesive properties and is easily injectable through a syringe.

The noted ranges may vary with the addition of various fillers, equivalents and other components or for other reasons.

The ratio between MKP (MKP equivalent, combination, and/or replacement) and the metal oxide (i.e., magnesia) in terms of the weight percent ratio can be between about 4:1 and 0.5:1 or between approximately 3:1 and 1:1. In the narrow range we speculate that the un-reacted magnesium is at least partly responsible for the in vivo expandability characteristics of the bio-adhesive.

Specifically the metal oxide (i.e., magnesium oxide) reacts with water and serum and in and around the living tissue to yield Mg(OH)₂ and magnesium salts. It has been found that some embodiments of the material generally expand to between 0.15 and 0.20 percent of volume during curing in moisture. The expansion of the material is believed to increase the adhesive characteristics of the material.

When potassium biphosphate (MKP) is used, sodium phosphate can also be added to the matrix in order to control the release of potentially dangerous ions to make the matrix more bio-compatible. When used for this purpose the sodium phosphate can be added in an amount sufficient to capture the desired amount of ions (i.e., potassium ions). The sodium phosphate (i.e., mono-sodium phosphate) is typically added up to about 20 weight percent, up to about 10 weight percent, or up to about 5 weight percent. Other sodium compounds may also prove helpful in this regard.

### V. TERTIARY CALCIUM PHOSPHATE

A tertiary calcium phosphate can be used in compositions of the invention as it increases both the bio-compatibility and bio-ab sorption of the biomaterial. Suitable tricalcium phosphates include α-Ca₃(PO₄)₂, β-Ca₃(PO₄)₂, and Ca₁₀(PO₄)₆(OH)₂. A preferred a tertiary calcium phosphate is a pharmaceutical or food grade tricalcium phosphate manufactured by Astaris (St. Louis, Mo.).

In addition to the tertiary calcium phosphate, other calcium-containing compounds can be added. In general, suitable calcium containing compounds include but are not limited to tricalcium phosphates, biphasic calcium phosphate, tetracalcium phosphate, amorphous calcium phosphate (“ACP”), CaSiO₃, oxyapatite (“OXA”), poorly crystalline apatite (“PCA”), octocalcium phosphate, dicalcium phosphate, dicalcium phosphate dihydrate, calcium metaphosphate, heptacalcium metaphosphate, calcium pyrophosphate and combinations thereof. Other calcium containing compounds include: ACP, dicalcium phosphate, CaSiO₃, dicalcium phosphate dihydrate and combinations thereof.

Calcium containing compounds increase the bio-compatibility and bioabsorption of the bio-adhesive. However, calcium containing compounds vary in their degrees of bioabsorption and biocompatibility. Some characteristics even vary within the various tricalcium phosphate compounds.

It may be advantageous to combine various calcium containing compounds to manipulate the bio-compatibility and bioabsorption characteristics of the material. For example Ca₁₀(PO₄)₆(OH)₂ (HA″) is stable in physiologic conditions and tends to be relatively poorly absorbed while β-Ca₃(PO₄)₂ is more readily absorbed. The two can be combined (bi-phasic calcium phosphate) to form a mixture having characteristics somewhere between HA and β-Ca₃(PO₄)₂.

### VI. REABSORBING AGENT

The reabsorbing agent may take a variety of forms. In one example, the reabsorbing agent comprises a sugar compound. The inventors have discovered that some sugar containing bio-materials have significant osteoproliferative properties as well as enhanced adhesive capabilities. It is believed that a sugar like sucrose may be used or replaced or supplemented with other sugars and sugar related compounds.

Suitable sugars or sugar related compounds include but are not limited to sugary materials such as: sugars, sugar derivatives (i.e., sugar alcohols, natural and artificial sweeteners (i.e., acesulfame-k, alitame, aspartame, cyclamate, neohesperidine, saccharin, sucralose and thaumatin), sugar acids, amino sugars, sugar polymers glycosaminoglycans, glycolipds, sugar polymers, sugar substitutes including sugar substitutes like sucralose (i.e., Splenda®, McNeil Nutritionals LLC, Ft. Washington, Pa.), corn syrup, honey, starches, and various carbohydrate containing substances.

Exemplary sugars include but are not limited to: sucrose, lactose, maltose, cellobiose, glucose, galactose, fructose, dextrose, mannose, arabinose, pentose, hexose. The sugar additive can be a polysaccharide or a disaccharide like sucrose. In one embodiment the sugar is combined with a flow agent like starch. An exemplary additive is approximately 97 weight percent sucrose and about 3 weight percent starch.

The sugar compound, like the other components, can be in a variety of forms including but not limited to dry forms (i.e., granules, powders etc.), aqueous forms, pastes, and gels. It may prove preferable to use a powdered form.

The inventor has shown that the invented sugar containing bio-material possess surprisingly good adhesive qualities. It is believed that the sugar may improve the physical (and possibly the chemical) bonding of the cement to objects. It is believed that the osteoproliferative properties of other bio-materials may possibly be enhanced by the addition of certain sugars (as disclosed herein). The addition of sugar compounds to prior art and future bio-materials such as PMMA and/or phosphate based materials may enhance their bone stimulating characteristics.

In another example, the reabsorbing agent comprises hydroxypropyl methyl cellulose (HPMC), carboxymethylcellulose (CMC), or a poloxamer. Example poloxamers include poloxamer 407 and poloxamer 188.

Surprisingly and unexpectedly, it was discovered that the compositions and methods of the invention provide improved reabsorption, improved porosity, and improved cohesion. This result was particularly surprising given recent studies showing the inability of calcium phosphate cements to be reabsorbed. The phosphate component of the composition allows for increased porosity. The increased porosity allows for a scaffold which provides a suitable microenvironment for the incorporation of cells or growth factors to regenerate damaged tissues and bony ingrowth. Scaffolds are generally highly porous with interconnected pore networks to facilitate nutrient and oxygen diffusion and waste removal. This scaffolding will also help with absorbability. The sugar component of the composition allows for adhesive properties. The adhesive properties are desired since the placement of this product is in bone void of some size. The adhesive qualities will allow the product to attach itself to both sides and create a scaffold to allow cells to regenerate bone.

### VII. BONE GRAFT MATERIAL

In one embodiment the composition of present invention provides a bone substitute and a platform for bone formation. An advantage of the substance is its gradual absorption by the body without rejection or reaction to contacted structures. A further advantage of the invented composition is its significant osteoproliferative properties. In fact, we have conducted studies that demonstrated that the composition of the invention enhanced bone formation to such a surprising degree that it appears that the composition is also osteoinductive, which is completely unexpected and unprecedented for a multi-purpose biomaterial without the use of growth factors. The bio-material is also believed to have micro and macro pores. Unexpectedly, initial tests have shown that the invented composition is capable of promoting motion preservation in a bone.

We have also observed that compositions of the present invention have unique bonding characteristics suitable for fixation of various medical prosthesis.

### VIII. ADDITIONAL EMBODIMENTS

The formulations disclosed herein may incorporate additional fillers, additives and supplementary materials. The supplementary materials may be added to the bio-material in varying amounts and in a variety of physical forms, dependent upon the anticipated use. The supplementary materials can be used to alter the bio-material in various ways.

Supplementary materials, additives, and fillers are preferably biocompatible and/or bioresorbable. In some cases it may be desirous for the material to be osteoconductive and/or osteoinductive as well. Suitable biocompatible supplementary materials include but are not limited to: bioactive glass compositions, calcium sulfates, coralline, polyatic polymers, peptides, fatty acids, collagen, glycogen, chitin, celluloses, starch, keratins, nucleic acids, glucosamine, chondroitin, and denatured and/or demineralized bone matrices, and other materials, agents, and grafts (autografts, allografts, xenografts). Other suitable supplementary materials are disclosed in U.S. Pat. No. 6,331,312 issued to Lee and U.S. Pat. No. 6,719,992 issued to Constanz, which are hereby incorporated by reference in their entireties.

In another embodiment of the invention the bio-material contains a radiographic material which allows for the imaging of the material in vivo. Suitable radiographic materials include but are not limited to barium oxide and titanium.

In yet another embodiment the invented bio-material contains a setting retarder or accelerant to regulate the setting time of the composition. Setting regulators are preferable biocompatible. Suitable retarders include but are not limited to sodium chloride, sodium fluosilicate, polyphosphate sodium, borate, boric acid, boric acid ester and combination thereof.

The disclosed bio-material may also be prepared with varying degrees of porosity. Controlling porosity can be accomplished through a variety of means including: controlling the particle size of the dry reactants, and chemical and physical etching and leaching. A preferred embodiment increases porosity of the bio-material by addition of 1-20 weight percent of a reabsorbing agent, preferably about 1-5 weight percent. Suitable reabsorbing agents include but are not limited: a sugar compound, hydroxypropyl methyl cellulose (HPMC), carboxymethylcellulose (CMC), a poloxamer, and combinations thereof.

The biomaterial may be used as delivery system by incorporating biologically active compounds into the bio-material (i.e., antibiotics, growth factors, cells, etc.). A porous bio-adhesive increases the effectiveness of such a delivery system.

Various antibiotics or other antibacterial and anti-viral compositions and agents can be added to the composition. The invented bio-material can act as a delivery device or the antibiotics can be added to protect against bacterial infection during surgery.

Cationic antibiotics, especially aminoglycosides and certain peptide antibiotics may be most desirable when incorporating drugs into the bio-material. Suitable aminoglycosides include but are not limited to: amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin. Using inorganic salts like sulfates, phosphates, hydrogenphosphates may be preferable, sulfates being the most preferable. Further information about using antibiotics and growth factors in bio-materials can be found in U.S. Pat. No. 6,485,754, issued to Wenz, which is hereby incorporated by reference in its entirety. Growth factors include but are not limited to growth factors like transforming growth factor TGF-β. Vancomycin and similar antibiotics can also be used.

The disclosed bio-material composition may also be seeded with various living cells or cell lines. Any known method for harvesting, maintaining and preparing cells may be employed. See U.S. Pat. No. 6,719,993 issued to Constanz, U.S. Pat. No. 6,585,992 issued to Pugh and, U.S. Pat. No. 6,544,290 issued to Lee.

We have shown that compositions of the invention are extremely useful as a scaffold for hard tissue growth and possibly soft tissue growth as well. In addition, tissue-producing and tissue-degrading cells may be added to the composition included but not limited to: osteocytes, osteoblasts, osteoclasts, chondrocytes, fibroblasts, cartilage producing cells, and stem cells. Methods of isolating and culturing such cells are well known in the art.

The composition of the invention can incorporated into an orthopedic kit comprising the material (i.e., MKP, metal oxide, calcium containing compounds etc.) in dry form, an activator solution (water or other aqueous solution), and any medical devices (i.e., syringes, knives, mixing materials, spatulas, etc.), implants, or other agents needed during an operation using the invented composition. The material and activator solution will preferably be present in a predetermined, optimized ratio. Other embodiments of such an orthopedic kit can also be envisioned. The biomaterial and other kit components are preferably sterilized by techniques well known in the art.

### IX. EXAMPLE METHOD

A method for back-filling a bone defect void using a bio-material with increased porosity and reabsorption characteristics is described herein. The method includes (a) removing a bone defect from a bone to create a void, (b) mixing a dry potassium phosphate based mixture with an aqueous solution to form a reabsorbable bio-material slurry, wherein the dry potassium phosphate based mixture comprises MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof, and (c) back-filling the void with the reabsorbable bio-material slurry, wherein the reabsorbable bio-material slurry increases osteoblast activity in the bone to help maintain the structure of the bone.

In such a method, the reabsorbable bio-material slurry turns to bone to provide improved bone structure in the bone. In contrast, traditional calcium-based bone fillers provide a scaffolding on which bone can grow, but do not turn into bone like the above-described composition. As such, the osteocytes in traditional calcium-based bone fillers run out and the bone filler deteriorates and is reabsorbed into the body. The advantage of the reabsorbable bio-material slurry described herein is that it actually turns into bone to thereby provide improved bone structure. In addition, the reabsorbable bio-material slurry described herein increases osteoblast activity in the bone due to the magnesium present in the reabsorbable bio-material slurry. Osteoblasts are the major cellular component of bone. Osteoblasts are specialized, terminally differentiated products of mesenchymal stem cells. They synthesize dense, crosslinked collagen and specialized proteins in much smaller quantities, including osteocalcin and osteopontin, which compose the organic matrix of bone. As such, the above method comprises a method for preserving bone comprising stimulating osteoblasts due to the magnesium present in the reabsorbable bio-material slurry to help maintain bone structure.

As discussed above, the method includes removing a bone defect from a bone to create a void. The bone defect may take a variety of forms. In particular, the bone defect may be selected from a group consisting of: a bone cyst, a bone marrow lesion, and an osteoporotic bone. A bone cyst is a fluid-filled hole that develops inside a bone. They mostly occur in children and young adults. Bone cysts do not usually cause any symptoms, they are not cancerous and they do not usually pose a serious threat to health. Bone marrow lesions (BMLs) or using older terminology “bone marrow edema” is characterized by excessive water signals in the marrow space on magnetic resonance imaging or ultrasound; BMLs constitute a central component of a wide variety of inflammatory and non-inflammatory rheumatologic conditions affecting the musculoskeletal system: BMLs are not only considered significant sources of pain but also linked to increased disease activity in many musculoskeletal conditions (for example, osteoarthritis, rheumatoid arthritis). The bone defects of the above method may be defects of the extremities and/or pelvic bone, as specific examples.

In one example, the method further includes positioning an anchor in the void prior to back-filling the void with the reabsorbable bio-material slurry. Such an anchor may provide additional structural support for the bone. The anchor may be a reabsorbable polymer material or a metal material. One example polymer material is poly-1 d-lactide (PLDLA).

### X. CONCLUSION

Having described the basic concept of the invention, it will be apparent to those skilled in the art that the foregoing detailed disclosure is intended to be presented by way of example only, and is not limiting. Various alterations, improvements, and modifications are intended to be suggested and are within the scope and spirit of the present invention. Additionally, the recited order of the elements or sequences, or the use of numbers, letters or other designations therefore, is not intended to limit the claimed processes to any order except as may be specified in the claims. Accordingly, the invention is limited only by the following claims and equivalents thereto.

All publications and patent documents cited in this application are incorporated by reference in their entirety for all purposes to the same extent as if each individual publication or patent document were so individually denoted and to the extent they are not inconsistent with the express teachings herein.

What is claimed is:

1. A bio-material composition, comprising:
(a) a dry potassium phosphate based mixture comprising: MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1; and
(b) an aqueous solution, wherein the dry potassium phosphate based mixture is mixed with the aqueous solution forming a reabsorbable bio-material slurry, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof, wherein the reabsorbing agent provides unexpected improved reabsorption, improved porosity, and improved cohesion of the bio-material composition.

2. The bio-material composition of claim 1, wherein the sugar compound selected from the group consisting of: sugars, sugar derivatives, sugar replacements, and combinations thereof.

3. The bio-material composition of claim 1, wherein the sugar compound is selected from a group consisting of: sugars, sugar alcohols, sugar acids, amino sugars, sugar polymers glycosaminoglycans, glycolipds, sugar substitutes, and combinations thereof.

4. The bio-material composition of claim 1, wherein the poloxamer is selected from a group consisting of: poloxamer 407, poloxamer 188, and combinations thereof.

5. The bio-material composition of claim 1, wherein the reabsorbing agent is between about 1-5 weight percent of the dry composition.

6. The bio-material composition of claim 1, wherein the reabsorbing agent is between about 1.5-2.5 weight percent of the dry composition.

7. The bio-material composition of claim 1, wherein the reabsorbing agent is between about 1.5-3 weight percent of the dry composition.

8. The bio-material composition of claim 1, wherein the reabsorbing agent is between about 1.5-3.5 weight percent of the dry composition.

9. The bio-material composition of claim 1, wherein the reabsorbing agent is between about 1.5-4 weight percent of the dry composition.

10. The bio-material composition of claim 1, wherein the reabsorbing agent is between about 1.5-4.5 weight percent of the dry composition.

11. The bio-material composition of claim 1, wherein the reabsorbing agent is about 2 weight percent of the dry composition.

12. The bio-material composition of claim 1, wherein reabsorbing agent is about 1.5-2 weight percent of the dry composition.

13. The bio-material composition of claim 1, wherein reabsorbing agent is about 1.5-2.5 weight percent of the dry composition.

14. The bio-material composition of claim 1, wherein the tertiary calcium phosphate is Ca₁₀(PO₄)₆(OH)₂.

15. The bio-material composition of claim 1, wherein the bio-material composition increases osteoblast activity in a bone.

16. The bio-material composition of claim 1, further comprising an antibiotic mixed with the dry potassium phosphate based mixture and the aqueous solution to form the reabsorbable bio-material slurry.

17. The bio-material composition of claim 16, wherein the antibiotic is selected from a group consisting of: gentamicin, tobramycin, and vancomycin.

18. A method for producing a bio-material with increased porosity and reabsorption characteristics, the method comprising:
supplying a dry potassium phosphate based mixture comprising: MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1;
mixing the dry potassium phosphate based mixture with an aqueous solution forming an reabsorbable bio-material slurry.

19. The method of claim 18, wherein the reabsorbing agent is selected from the group consisting of a sugar compound, hydroxypropyl methyl cellulose (HPMC), carboxymethylcellulose (CMC), a poloxamer, and combinations thereof.

20. The method of claim 18, wherein the reabsorbing agent is between about 1-10 weight percent of the dry composition.

21. A method for back-filling a bone defect void using a bio-material with increased porosity and reabsorption characteristics, the method comprising:
removing a bone defect from a bone to create a void;
mixing a dry potassium phosphate based mixture with an aqueous solution to form a reabsorbable bio-material slurry, wherein the dry potassium phosphate based mixture comprises MgO, monobasic potassium phosphate, monobasic sodium phosphate, a reabsorbing agent, and a tertiary calcium phosphate, wherein the weight percent ratio of monobasic potassium phosphate to metal oxide is between about 3:1 and 1:1, wherein the reabsorbing agent is between about 1-10 weight percent of the dry composition, and wherein the reabsorbing agent is selected from the group consisting of a sugar compound, hydroxypropyl methyl cellulose (HPMC), carboxymethylcellulose (CMC), a poloxamer, and combinations thereof; and
back-filling the void with the reabsorbable bio-material slurry, wherein the reabsorbable bio-material slurry increases osteoblast activity in the bone to help maintain the structure of the bone.

22. The method of claim 21, wherein the reabsorbable bio-material slurry turns to bone to provide bone structure in the bone.

23. The method of claim 21, wherein the bone defect is selected from a group consisting of: a bone cyst, a bone marrow lesion, and an osteoporotic bone.

24. The method of claim 21, further comprising positioning an anchor in the void prior to back-filling the void with the reabsorbable bio-material slurry, wherein the anchor provides additional structural support for the bone.

25. The method of claim 24, wherein the anchor comprises a polymer or a metal.
